(19) United States
(12) Patent Application Publication
(10) Pub. No.: US 2010/0092573 A1
(43) Pub. Date: Apr. 15, 2010

(54) HEMOSTATIC BIO-MATERIAL COMPOSITION AND METHOD

(76) Inventors: Thomas Lally, Chicago, IL (US); Sunil Prasad, Chicago, IL (US)

Correspondence Address: THOMAS LALLY, 603 MALLARD LANE, OAK BROOK, IL, 60523, US

(21) Appl. No.: 12/450,170

(22) PCT Filed: Mar. 12, 2008
(86) PCT No.: PCT/US08/03230

§ 371 Date: Sep. 14, 2009

Related U.S. Application Data
(60) Provisional application No. 60/906,458, filed on Mar. 12, 2007.

(51) Int. Cl. A61K 33/42; A61P 7/00

(52) U.S. Cl. 424/602

(57) ABSTRACT

The present invention relates to a haemostatic bio-material composition and method for achieving hemostasis. The method for providing hemostasis generally comprises: supplying a dry potassium phosphate based hemostat mixture comprising: monobasic potassium phosphate, a metal oxide, and a tertiary calcium phosphate, wherein the weight percent ratio of monobasic potassium phosphate to metal oxide is between about 3:1 and 1:1; mixing the dry potassium phosphate based hemostat mixture with an aqueous solution forming an activated hemostat slurry; applying an hemostasis-promoting amount of the activated potassium phosphate based hemostat slurry to a site of bleeding; wherein the site of bleeding is in, on, or proximate to bone.

### TECHNICAL FIELD

The present invention relates to method for achieving hemostasis. More specifically one preferred embodiment of the invention relates to a hemostatic composition and method for providing hemostasis.

### BACKGROUND OF THE INVENTION

A variety of methods and substances have been used by medical professionals over the years to control bleeding from hard tissues such as bone. Collagen, oxidized cellulose, thrombin, and other materials and agents have been used, but each of these compositions has its limitations.

Collagen is a water insoluble fiber that has inherent haemostatic properties. Although collagen and collagen containing products can be effective hemostats they are often difficult to work with and/or fail to make good contact with the wound site.

Oxidized cellulose is a solid bio-absorbable composition which is soluble in certain bodily fluids from wounds. Oxidized cellulose forms a sticky mass which readily adheres to wound surfaces, however, its insolubility in water is a major drawback.

Thrombin is a very well known clotting agent but can cause allergic reactions in a certain percentage of the population. Thrombin also requires mixing with another agent, such as cryoprecipitate or calcium, and also requires a carrier medium.

What is needed is a haemostatic composition and method that avoids some or all of the disadvantages of the pre-existing haemostatic compositions and methods discussed above, that is easy to handle and work with, that adheres well to cut bone surfaces, bone fractures (or other wounds), and that provides an effective level of hemostasis, with little or no systemic or local adverse effect. Preferably the composition would be osteoproliferative to enhance bone healing when desired. It would also be preferable if the composition protected against infection.

### SUMMARY OF THE INVENTION

The present invention relates to a haemostatic bio-material composition and method for achieving hemostasis. The method for providing hemostasis generally comprises: supplying a dry potassium phosphate based hemostat mixture comprising: monobasic potassium phosphate, a metal oxide, and a tertiary calcium phosphate, wherein the weight percent ratio of monobasic potassium phosphate to metal oxide is about 3:1 and 1:1;

mixing the dry potassium phosphate based hemostat mixture with an aqueous solution forming an activated hemostat slurry; applying a hemostasis-promoting amount of the activated potassium phosphate based hemostat slurry to a site of bleeding; wherein the site of bleeding is in, on, or proximate to bone. In one or more preferred embodiments the dry mixture further comprises: a sugar (or sugar derivate/replacement) and/or monosodium phosphate.

The invented haemostatic composition has excellent adhesive properties, is easy to mix and apply and provides very effective haemostasis to prevent blood loss from exposed bone surfaces without significant adverse reactions. The invented composition may also be capable of being used as a haemostatic composition for other injuries.

One or more antibiotics or other antibacterial agents can be incorporated into the hemostat mixture to protect against bacterial infections which often result from certain common surgeries and or injuries that result in lose of blood due to exposure of hard tissues such as bone.

### DETAILED DISCLOSURE OF THE INVENTION

The present invention describes a method for providing hemostasis. The present invention is particularly well suited for use with sternotomy or other bone incisions, but can also be used on other cut or exposed bone surfaces and may be possible to use in other injuries including cuts, incisions and other defects in hard tissue.

In a preferred embodiment, the thickness of the haemostatic paste composition is such as to easily spread on the cut bone surfaces or exposed bone surface to halt bleeding. The composition can be directly applied to the exposed bone surface or other wound to stop bleeding.

The hemostat composition is not removed from the cut bone surface or exposed bone prior to closing the surgical site. The composition adheres to the bone surface and quickly slows and even stops bleeding from the site. Typically, the material hardens after application. The invented composition has been shown in earlier studies to be osteoproliferative and may stimulate the formation of bone around the wound site. Over time the composition has previously been shown to be bioabsorbable.

### DEFINITIONS

“Osteoconductive” is the ability of material to serves as a scaffold for viable bone growth and healing.

“Osteoinductive” refers to the capacity to stimulate or induce bone growth.

“Biocompatible” refers to a material that elicits no significant undesirable response in the recipient.

“Bioresorbable” is defined as a material's ability to be resorbed in-vivo through bodily processes. The resorbed material may be used the recipients body or may be excreted.

“Hemostasis” refers to halting or stopping bleeding in an animal or human. A hemostat or haemostat refers to a method, apparatus or composition that is employed to create hemostasis.

As used herein, a “hemostasis-promoting amount” is the amount effective to accelerate clot formation at an interface between a surface (e.g., of a wound or lesion) and the hemostatic composition.

“Prepared Cells” are defined as any preparation of living cells including but not limited to tissues, cell lines, transformed cells, and host cells. The cells are preferably autologous but can also be xenogeneic, allogeneic, and syngeneic.

### BEST MODE FOR CARRYING OUT THE INVENTION

The present invention describes a method for providing hemostasis generally comprising: supplying a dry potassium phosphate based hemostat mixture comprising: monobasic potassium phosphate, a metal oxide, and a tertiary calcium phosphate, wherein the ratio of monobasic potassium phosphate to metal oxide is about 3:1 and 1:1; mixing the dry potassium phosphate based hemostat mixture with an aqueous solution forming an activated hemostat slurry; applying an hemostasis-promoting amount of the activated potassium phosphate based hemostat slurry to a site of bleeding; wherein the site of bleeding is in, on, or proximate to bone.

Generally, the hemostat slurry is derived from a mixing a dry hemostat mixture with an aqueous solution as described below.

### Preparing/Supplying the Dry Hemostat Mixture

A salient aspect of the invention is the dry hemostat mixture. The dry mixture generally comprises: monobasic potassium phosphate (mono-potassium phosphate also known as MKP), a metal oxide, and a tertiary calcium phosphate, wherein the weight percent ratio of monobasic potassium phosphate to metal oxide is between about 3:1 and 1:1. In one or more preferred embodiments the dry mixture also comprises a sugar and/or a mono-sodium phosphate. It may be preferable to produce the dry hemostat mixture in advance. After it is prepared it should be stored in a sterile environment and more preferably a sterile and sealed container or packaging.

The dry components of the hemostat can be mixed using a variety of methods including hand mixing or machine mixing. One method for mixing, sizing and homogenizing the various powders is via vibratory milling. Another homogenization method utilizes a ribbon mixer wherein the particles are ground to a fine size. It may be preferable to mix the dry components again on-site before the addition of the activating aqueous solution.

A metal oxide powder is a salient ingredient in the dry hemostat mixture. Optionally, the oxide is subjected to a calcinated process. Calcination durations and temperatures are determined empirically, depending on the final characteristics and setting times desired. In some embodiments calcination temperatures of up to about 1300° C. for up to several hours are used, although calcination can be varied.

Dry compounds are disclosed herein, however, it may be possible to substitute aqueous versions (or other forms i.e. gels etc) of the components in certain situations. Generally, pharmaceutical grade compounds are utilized.

Sterilization of the components, utensils, solutions etc. used to make and apply the hemostat may be required using suitable sterilization techniques known in the art including but not limited to chemical sterilization techniques, such as gassing with ethylene oxide, and sterilization by means of high-energy radiation, usually y radiation or β radiation.

Details of the dry mixture composition is described below in detail.

### Forming an Activated Hemostat Mixture

The hemostat mixture is preferably activated on-site. The supplied dry hemostat mixture is mixed with an aqueous solution in a sterile mixing vessel to a form an activated hemostat slurry. The sterile water (or other sterile aqueous solution i.e. slight saline solution) is generally added up to about 40% of the dry weight although the amount of water can be adjusted to form a bio-material of varying viscosity. As discussed below it is preferable to produce a paste like consistency, it was found that the addition of between about 20-30 weight percent aqueous solution was generally suitable to obtain such consistency dependent upon conditions.

In a preferred embodiment, the mixing vessel and mixing utensil are sterilized prior to use. Various mixing vessels can be used including but not limited to a sterile medicine cup, bowl, dish, basin or other sterile container.

The activated hemostat slurry is typically hand mixed for between about 1-10 minutes, although mixing times can be adjusted depending upon conditions and mixing means. Mixing can be achieved by a variety of techniques used in the art including hand and electric/automated mixing. One preferred method is to hand mix with a sterile spatula or other mixture utensil.

It may be possible to mix the slurry using manual hand mixers like the Mixevac III from Stryker (Kalamazoo, Mich.) or an electric bone mixer like the Cemex Automatic Mixer from Exactech (Gainesville, Fla.).

The hemostat can be created in injectable, paste, putty and other forms. Since the slurry is produced at the user site the consistency of the material can be manipulated by varying the amount of water added to the dry mixture. Increasing the water content generally increases the flowability while decreasing the water content tends to thicken the slurry.

Working times can be increased or decreased by varying the temperatures of bio-material components. Higher temperature components tend to react and set quicker than cooler components. Thus regulating the temperature of the water (or other reactants) can be an effective way to regulate working time.

The inventors have found that the use of a phosphoric acid instead of water increases the bonding strength of the material. The molarity of the phosphoric acid can vary, as long as the eventual pH of the slurry is not hazardous to the patient, or contraindicative to healing.

### Applying the Activated Hemostat to the Site of Bleeding

Once the activated slurry has been formed the activated hemostat is applied to (and optionally also around) the site of bleeding. The slurry can be applied to the site in a number of ways including but not limited to spreading a hemostasis-promoting amount of the material to the site using a sterile spatula, tongue blade, knife or other sterile implement useful for spreading a paste or putty-like material. It is generally preferable to use a relatively thick consistency like a paste or putty when applying the hemostat, since such consistencies tend to stick to bone surface more easily than thinner ones. If an injectable formation is desired, it can be applied using a syringe or other similar device.

Exemplary formulations of the dry hemostat mixture include the following:

Formulation I*

| | |
|---|---|
| Mono-potassium phosphate (i.e. KH₂PO₄) | 61% |
| MgO (calcined) | 31% |
| Ca₁₀(PO₄)₆(OH)₂ | 4% |
| Sucrose C₁₂H₂₂O₁₁ (powder) | 4% |

*All values are weight percentages

Water is added up to about 40 weight percent of the dry formulation, preferably between about 20-35 weight percent.

Formulation II*

| | |
|---|---|
| KH₂PO₄ | 54% |
| MgO (calcined) | 33% |
| Calcium-containing compound | 9% (whereby the compound is |
| | Ca₁₀(PO₄)₆(OH)₂) |
| Sucrose C₁₂H₂₂O₁₁ (powder) | 4% |

*All values are weight percentages

Water is added up to about 40 weight percent of the dry formulation, preferably between about 20-35 weight percent.

Formulation III*

| | |
|---|---|
| KH₂PO₄ | 44% |
| MgO (calcined) | 44% |
| Calcium-containing compound | 8% (whereby the compound is |
| | Ca₁₀(PO₄)₆(OH)₂ or CaSiO₃, |
| Sucrose C₁₂H₂₂O₁₁ (powder) | 4% |

*All values are weight percentages

Water is added up to about 40 weight percent of the dry formulation, preferably between about 20-35 weight percent.

Formulation IV*

| | |
|---|---|
| Potassium phosphate (i.e. KH₂PO₄) | 44% |
| MgO (calcined) | 41% |
| Ca₁₀(PO₄)₆(OH)₂ | 8% |
| Sucrose C₁₂H₂₂O₁₁ (powder) | 4% |
| Mono-sodium phosphate (MSP) | 3% |

*All values are weight percentages

Water is added up to about 40 weight percent of the dry formulation, preferably between about 20-35 weight percent, more preferably between about 28-32 weight percent.

Formulation V*

| | |
|---|---|
| KH₂PO₄ (MKP) | 45% |
| MgO (calcined) | 45% |
| Calcium-containing compound | 9% |
| Sucrose C₁₂H₂₂O₁₁ (powder) | 1% |

*All values are weight percentages

Water is added up to about 40 weight percent of the dry formulation, preferably between about 20-35 weight percent.

Formulation VI*

| | |
|---|---|
| KH₂PO₄ | 45% |
| MgO (calcined) | 45% |
| Ca₁₀(PO₄)₆(OH)₂ | 8% |
| Sucralose | 2% |

*All values are weight percentages

Water is added up to about 40 weight percent of the dry formulation, preferably between 20-35 weight percent.

Formulation VII*

| | |
|---|---|
| KH₂PO₄ | 61% |
| MgO (calcined) | 32% |
| Ca₁₀(PO₄)₆(OH)₂ | 4% |
| Collagen | 1.5% |
| α-Ca₃(PO₄)₂ | 1.5% |

*All values are weight percentages

Water is added up to about 40 weight percent of the dry formulation, preferably between 20-35 weight percent.

Formulation VIII*

| | |
|---|---|
| KH₂PO₄ | 50% |
| MgO (calcined) | 35% |
| Ca₁₀(PO₄)₆(OH)₂ | 7% |
| β-Ca₃(PO₄)₂ | 3% |
| Dextrose | 5 |

*All values are weight percentages

Water is added up to about 40 weight percent of the dry formulation, preferably between 20-35 weight percent.

Formulation IX*

| | |
|---|---|
| KH₂PO₄ | 54% |
| Phosphoric Acid | 4% |
| Metal oxide | 32% (wherein the metal oxide is MgO, |
| | ZrO, FeO or combination thereof), |
| Ca₁₀(PO₄)₈(OH)₂) | 7% |
| Thrombin | 3% |

*All values are weight percentages

Water is added up to about 40 weight percent of the dry formulation, preferably between 20-35 weight percent.

Formulation X*

| | |
|---|---|
| KH₂PO₄ | 45% |
| MgO (calcined) | 45% |
| Ca₁₀(PO₄)₆(OH)₂ | 10% |

Water is added up to about 40 weight percent of the dry formulation, preferably between 20-35 weight percent.

While the above formulations and weight percents are the preferred proportions, a range of dry constituents can also be used. For example, a suitable range for the potassium phosphate (i.e. MKP) is generally between about 20-70 weight percent, preferably between about 40-65 weight percent. In some situations and/or embodiments it is preferable to use the potassium phosphate at a range between about 40-50 weight.

A suitable range for the metal oxide (i.e. MgO) is generally between about 10-60, preferably between 10-50, and even more preferably between 30-50 weight percent. In some situations and/or embodiments it maybe preferable to use between about 35 and 50 weight percent.

Calcium containing compounds (i.e. tertiary calcium phosphates) can be added in various weight percentages. The calcium containing compound(s) is preferably added at about 1-15 weight percent, more preferably between about 1-10 weight percent. Higher percentages can be employed in certain situations.

Sugars (and/or other carbohydrate containing substances) are generally present at weight percent between 0.5 and 20, preferably about 0.5-10 weight percent of the dry composition.

Typically the antibiotic, antibacterial or antiviral agent is added at a weight percent of less than about 20 weight percent of the dry composition, preferably between about 0.5 and 10 weight percent, more preferably between about 1 and 5 weight percent.

Water (or another aqueous solution) can be added in a large range of weight percents generally ranging from about 15-40 weight percent, preferably between about 20-30 weight percent and even more preferably about 25 weight percent. It was found that a saline solution may be used. An exemplary saline solution is a 0.9% saline solution.

For some embodiments (i.e. formula III) it has been found that adding water at a weight percent of about 37 weight percent produces a creamy textured material that is extremely easy to work with has excellent adhesive properties and is easily injectable through a syringe.

The noted ranges may vary with the addition of various fillers, equivalents and other components or for other reasons.

A salient feature of the present invention is the ratio between MKP (MKP equivalent, combination, and/or replacement) and the metal oxide. A preferred embodiment has a weight percent ratio between MKP and MgO between about 4:1 and 0.5:1, more preferably between approximately 3:1 and 1:1. In such a preferred embodiment the inventor surmises that the un-reacted magnesium is at least partly responsible for the in vivo expandability characteristics of the bio-adhesive.

Specifically the metal oxide (i.e. magnesium oxide) reacts with water and serum and in and around the living tissue to yield Mg(OH)₂ and magnesium salts. It has been found that some embodiments of the material generally expand to between 0.15 and 0.20 percent of volume during curing in moisture. The expansion of the material is believed to increase the adhesive characteristics of the material

MgO is the preferred metal oxide (metal hydroxide or other equivalent), however, other metal oxides can be utilized in place of or in addition to MgO, including but not limited to: FeO, Al(OH)₃, Fe₂O₃, Fe₃O₄, ZrO, and Zr(OH)₄, zinc oxides and hydroxides, calcium oxide and hydroxides and other metal, oxides, hydroxides and equivalents and combinations thereof.

MKP is salient aspect of the invention. Inventor has discovered that a sodium phosphate can also be added to the matrix in order to control the release of potentially dangerous potassium ions to make the matrix more bio-compatible. When used for this purpose the sodium phosphate can be added in an amount sufficient to capture the desired amount of ions (i.e. potassium ions). The sodium phosphate (i.e. mono-sodium phosphate) is typically added up top about 20 weight percent, preferably up to about 10 weight percent, and even more preferably up to about 5 weight percent. Other sodium compounds may also prove helpful in this regard.

### Tertiary Calcium Phosphate

A tertiary calcium phosphate is essential to the invention as it increases both the bio-compatibility and bio-absorption of the biomaterial. Suitable tricalcium phosphates include α-Ca₃(PO₄)₂, β-Ca₃(PO₄)₂, and Ca₁₀(PO₄)₆(OH)₂. A preferred a tertiary calcium phosphate is a pharmaceutical or food grade tricalcium phosphate manufactured by Astaris (St. Louis, Mo.).

In addition to the tertiary calcium phosphate other calcium containing compounds can be added. In general, suitable calcium containing compounds include but are not limited to: tricalcium phosphates, biphasic calcium phosphate, tetracalcium phosphate, amorphous calcium phosphate (“ACP”), CaSiO₃, oxyapatite (“OXA”), poorly crystalline apatite (“PCA”), octocalcium phosphate, dicalcium phosphate, dicalcium phosphate dihydrate, calcium metaphosphate, heptacalcium metaphosphate, calcium pyrophosphate and combinations thereof. Other calcium containing compounds include: ACP, dicalcium phosphate, CaSiO₃, dicalcium phosphate dihydrate and combinations thereof.

Calcium containing compounds increase the bio-compatibility and bioabsorption of the bio-adhesive. However, calcium containing compounds vary in their degrees of bioabsorption and biocompatibility. Some characteristics even vary within the various tricalcium phosphate compounds.

It may be advantageous to combine various calcium containing compounds to manipulate the bio-compatibility and bioabsorption characteristics of the material. For example Ca₁₀(PO₄)₆(OH)₂ (HA″) is stable in physiologic conditions and tends to be relatively poorly absorbed while β-Ca₃(PO₄)₂ is more readily absorbed. The two can be combined (i.e. bi-phasic calcium phosphate) to form a mixture having characteristics somewhere between HA and β-Ca₃(PO₄)₂. A number of calcium containing compound combinations can be envisioned.

### Sugars, Sugar Substitutes, Sweeteners, Carbohydrates and Equivalents

A salient aspect of a preferred embodiment is the incorporation of at least one sugar or sugar like substance to the bio-material matrix. Inventor discovered that some sugar containing bio-materials have significant osteoproliferative properties as well as enhanced adhesive capabilities. It is believed that a sugar like sucrose may be replaced or supplemented with other sugars and sugar related compounds.

Suitable sugars or sugar related compounds include but are not limited to sugary materials such as: sugars, sugar derivatives (i.e. sugar alcohols, natural and artificial sweeteners (i.e. acesulfame-k, alitame, aspartame, cyclamate, neohesperidine, saccharin, sucralose and thaumatin), sugar acids, amino sugars, sugar polymers glycosaminoglycans, glycolipids, sugar polymers, sugar substitutes including sugar substitutes like sucralose (i.e. Splenda®, McNeil Nutritionals LLC, Ft. Washington, Pa.), corn syrup, honey, starches, and various carbohydrate containing substances.

Exemplary sugars include but are not limited to: sucrose, lactose, maltose, cellobiose, glucose, galactose, fructose, dextrose, mannose, arabinose, pentose, hexose. Preferably the sugar additive is a polysaccharide, more preferably a disaccharide like sucrose. In one embodiment sugar combined with a flow agent like starch. An exemplary additive is approximately 97 weight percent sucrose and about 3 weight percent starch.

The sugar compound, like the other components, can be in a variety of forms including but not limited to dry forms (i.e. granules, powders etc.), aqueous forms, pastes, and gels. It may prove preferable to use a powdered form.

The inventor has shown that the invented sugar containing bio-material possess surprisingly good adhesive qualities. It is believed that the sugar may improve the physical (and possibly the chemical) bonding of the cement to objects.

Surprisingly and unexpectedly, it was discovered that a sugar containing composition greatly enhanced formation of new bone. It is believed that the sugar and/or other compounds of the composition provide near ideal conditions for new bone formation.

### Bone Graft Material

In one embodiment the composition of present invention provides a bone substitute and a platform for bone formation. An advantage of the substance is its gradual absorption by the body without rejection or reaction to contacted structures. A further advantage of the invented composition is its significant osteoproliferative properties. In fact, in studies the invented composition enhanced bone formation to such a surprising degree, so much so that it is believed that the composition may also be osteoinductive which is completely unexpected and unprecedented for a multi-purpose biomaterial without the use of growth factors. The bio-material is also believed to have micro and macro pores.

### Additional Embodiments

The formulations disclosed herein may incorporate additional fillers, additives and supplementary materials. The supplementary materials may be added to the bio-material in varying amounts and in a variety of physical forms, dependent upon the anticipated use. The supplementary materials can be used to alter the bio-material in various ways.

Supplementary materials, additives, and fillers are preferably biocompatible and/or bioresorbable. In some cases it may be desirous for the material to be osteoconductive and/or osteoinductive as well. Suitable biocompatible supplementary materials include but are not limited to: bioactive glass compositions, calcium sulfates, coralline, polyatic polymers, peptides, fatty acids, collagen, glycogen, chitin, celluloses, starch, keratins, nucleic acids, glucosamine, chondroitin, and denatured and/or demineralized bone matrices, and other materials, agents, and grafts (autografts, allografts, xenografts). Other suitable supplementary materials are disclosed in U.S. Pat. No. 6,331,312 issued to Lee and U.S. Pat. No. 6,719,992 issued to Constanz, which are hereby incorporated by reference in their entireties.

The invention also imagines the addition of other hemostatic agents to the composition including but not limited to: collagen, collagen protein, thrombin, oxidized cellulose and combinations thereof.

In another embodiment of the invention the bio-material contains a radiographic material which allows for the imaging of the material in vivo. Suitable radiographic materials include but are not limited to barium oxide and titanium.

In yet another embodiment the invented bio-material contains a setting retarder or accelerant to regulate the setting time of the composition. Setting regulators are preferable biocompatible. Suitable retarders include but are not limited to sodium chloride, sodium fluosilicate, polyphosphate sodium, borate, boric acid, boric acid ester and combination thereof.

The disclosed bio-material may also be prepared with varying degrees of porosity. Controlling porosity can be accomplished through a variety of means including: controlling the particle size of the dry reactants, and chemical and physical etching and leaching. A preferred embodiment increases porosity of the bio-material by addition of 1-20 weight percent of an aerating agent, preferably about 1-5 weight percent. Suitable aerating agents include but are not limited: carbonates and bicarbonates such as: calcium carbonate, sodium carbonate, sodium bicarbonate, calcium bicarbonate, baking soda, baking powder, and combinations thereof.

The biomaterial may be used as delivery system by incorporating biologically active compounds into the bio-material (i.e. antibiotics, growth factors, cell etc.). A porous bio-adhesive increases the effectiveness of such a delivery system.

Various antibiotics or other antibacterial and anti-viral compositions and agents can be added to the composition. The invented bio-material can act as a delivery device or the antibiotics can be added to protect against bacterial infection during surgery.

Cationic antibiotics, especially aminoglycosides and certain peptide antibiotics may be most desirable when incorporating drugs into the bio-material. Suitable aminoglycosides include but are not limited to: amikacin, butirosin, dideoxykanamycin, fortimycin, gentamycin, kanamycin, lividomycin, neomycin, netilmicin, ribostamycin, sagamycin, seldomycin and epimers thereof, sisomycin, sorbistin, spectinomycin and tobramycin. Using inorganic salts like sulfates, phosphates, hydrogenphosphates maybe preferable, sulfates being the most preferable. Further information about using antibiotics and growth factors in bio-materials can be found in U.S. Pat. No. 6,485,754, issued to Wenz, which is hereby incorporated by reference in its entirety. Growth factors include but are not limited to growth factors like transforming growth factor TGF-β. Vancomycin and similar antibiotics can also be used.

The disclosed bio-material composition may also be seeded with various living cells or cell lines. Any known method for harvesting, maintaining and preparing cells may be employed. See U.S. Pat. Nos. 6,719,993 issued to Constanz, 6,585,992 issued to Pugh and, 6,544,290 issued to Lee.

One embodiment of the invention has been shown to be extremely useful as a scaffold for hard tissue growth and possibly soft tissue growth as well. In addition, tissue-producing and tissue-degrading cells may be added to the composition included but not limited to: osteocytes, osteoblasts, osteoclasts, chondrocytes, fibroblasts, cartilage producing cells, and stem cells. Methods of isolating and culturing such cells are well known in the art.

The invented composition can incorporated into an orthopedic kit comprising: the material (i.e. MKP, metal oxide, calcium containing compounds etc.) in dry form, an activator solution (water or other aqueous solution), and any medical devices (i.e. syringes, knives, mixing materials, spatulas, etc.), implants, or other agents needed during an operation using the invented composition. The material and activator solution will preferably be present in a predetermined, optimized ratio. Other embodiments of such an orthopedic kit can also be envisioned. The biomaterial and other kit components are preferably sterilized by techniques well known in the art.

### Hemostat Test Data

TEST FACILITY: NAMSA 6750 Wales Road Northwood, Oreg. 43619
NAMSA SPONSOR: Tom Lally; Bone Solutions, Inc. 603 Mallard Lane Oak Brook, Ill. 60523
STUDY TITLE: Evaluation of Hemostasis Following Application of Test Article—Pilot Study
TEST ARTICLE: OsteoCrete (See, Formulation IV)
IDENTIFICATION NO.: Lot: EWO BONOI-004/EWO-BONOI-003
PEOPLE>SCIENCE>SOLUTIONS P.O. No. 23

Control of bleeding at a surgical site is important. Hemostasis can be achieved by mechanically blocking or plugging bleeding vessels or through stimulation of the clotting cascade. The test article, OsteoCrete, Lot: EWO BONO1-004/EWO-BONO1-003, has been previously evaluated as a bone void filler. Due to its physical and mechanical properties, it may have applications as a hemostatic agent. The objective of the study is to evaluate the test article as a hemostatic agent. The test article was evaluated in two types of tissue, bone and organ parenchyma. Two domestic pigs had surgical defects made to the sternum, spleen, and liver. The test article was applied to these defects and the time to hemostasis was recorded. Under the conditions of the study, the OsteoCrete would be an acceptable hemostatic agent in bone tissue. The test material did not show evidence of being an effective hemostatic agent in the organ parenchyma.
Study and Supervisory Personnel: Theresa A. Ford-Wells, B.S., RVT, A.A.S., ALAT Christina R. Young, RVT, A.A.S., Joseph W. Carraway, D.V.M., M.S.; Vanessa K. Mock, RVT, A.A.S., Amy J. Debo, RVT, A.A.S., Lisa A. Severhof, B.A.
Contributing Scientist: Amanda L. Johnson, Bone Solutions, Inc.
Approved by: Joseph W. Carraway, D.V.M., M.S.-Director of Toxicology and Michelle E. Longstreet, B.S.-Study Director

### 1. Introduction

### Background

Control of bleeding at a surgical site is important. Hemostasis can be achieved by mechanically blocking or plugging bleeding vessels or through stimulation of the clotting cascade. The test article has been previously evaluated as a bone void filler. Due to its physical and mechanical properties, it may have applications as a hemostatic agent.

### Purpose

The objective of the study is to evaluate the test article as a hemostatic agent. The test article was evaluated in two types of tissue, bone and organ parenchyma. Two domestic pigs had surgical defects made to the sternum, spleen and liver. The test article was applied to these defects and the time to hemostasis was recorded.

### Testing Guidelines

There are no specific testing guidelines that define the methods used for evaluating the effectiveness of hemostatic agents. However, ISO 10993: Biological Evaluation of Medical Devices, Part 4: Selection of tests for interactions with blood provides general testing requirements for any material with blood interactions. Dates: The test article was received on Jun. 27, 2007. The surgery took place on Jul. 2, 2007.

### Duplication of Experimental Work

By signature on the protocol, the sponsor confirmed that the conduct of this study did not unnecessarily duplicate previous experiments.

### 2. Materials

The test article provided by the sponsor was identified and handled as follows:
Test Article: OsteoCrete
Identification No.: Lot: EWO BONOI-004/EWO-BONOI-003
Storage Conditions: Room Temperature
Preparation: The test article was provided sterile. The OsteoCrete was kept at room temperature prior to mixing. Just prior to application, 6 ml of the sponsor provided modified saline solution were poured into a sterile mixing bowl. The packet of OsteoCrete bone powder was opened and 25 grams were added to the bowl. The powder and saline solution were mixed vigorously for 2 minutes with the provided spatula until a paste-like consistency was achieved.

### 3. Test System

Species: Swine (Sus scrofa domesticus
Source: Michael Fanning Farms
Sex: Male
Body Weight Range: 54 kg to 54 kg at time of surgery
Age: Approximately 4 months at time of surgery
Acclimation Period: Minimum 7 days
Number of Animals: two
Identification Method: Ear tag

### Justification of Test System

Domestic swine have been used historically to study hemorrhage including the pathophysiology and treatment. The hemodynamic effects of volume hemorrhage in swine are well documented and similar to that of humans. The vascular anatomy, size, and blood volume of the animals are similar to those of an adult human and will allow for evaluation of the hemostatic strategies of the article in a clinical like application.

### 4. Animal Management

Husbandly: Conditions conformed to Standard Operating Procedures that are based on the “Guide for the Care and Use of Laboratory Animals.”
Food: A commercially available, diy, swine diet was provided daily.
Water: Potable water was provided ad libitum through species appropriate water containers or delivered through an automatic watering system
Contaminants: Reasonably expected contaminants in feed or water supplies did not have the potential to influence the outcome of this test.
Housing: Animals were individually housed in pens identified by a card indicating the lab number, animal number, test code, sex, and first treatment date.
Environment The room temperature was monitored daily. The recommended temperature range for the room was 61-81 oF. The room humidity was monitored daily. The humidity range for the room was 30-70%. The light cycle was controlled using an automatic timer (\2 hours light, 12 hours dark). Accreditation: NAMSA is an AAALAC International accredited facility and is registered with the United States Department of Agriculture. Additionally, NAMSA maintains an approved Animal Welfare Assurance on file with the National Institutes of Health, Office for Laboratory Animal Welfare.
Personnel: Associates involved were appropriately qualified and trained.
Selection: Healthy, previously unused animals were selected.
Sedation, Analgesia or Anesthesia: Sedation, analgesia or anesthesia was necessary during the routine course of this procedure.
Veterinary Care: In the unlikely event that an animal became injured, ill, or moribund, care was conducted in accordance with current veterinary medical practice. If warranted for humane reasons, euthanasia was conducted in accordance with the current report of the American Veterinary Medical Association's Panel on Euthanasia. The objective of the study will be given due consideration in any decision and the study sponsor will be advised.
IACUC: Review and approval by a Divisional NAMSA Institutional Animal Care and Use Committee (IACUC) is necessary prior to conduct of the study. Any significant changes to this protocol must be approved by the IACUC.

### 5. Methods

### Pre-operative Procedure

On the day prior to scheduled surgery, food was withheld overnight from each animal. On the following day, each animal was weighed and general anesthesia was induced with an intramuscular injection of a combination Tiletamine/zolazepam at 4.4 mg/kg and xylazine at 2.2 mg/kg. An intravenous catheter was placed in the lateral ear vein and 0.9% saline administered at a maintenance level and as needed to maintain blood pressure. A non-medicated ophthalmic ointment was applied to eyes of each animal to protect the corneas from drying. The vital signs (temperature, heart rate, EKG, respiration rate, and PO,) for each animal were monitored during the procedure. Blood pressure was monitored indirectly with a cuff placed on one limb. Each animal was intubated and placed on isoflurane inhalant anesthetic for continued general anesthesia. The animal was placed on positive pressure ventilation once the thoracic cavity had been entered. The hair on the inguinal region, abdomen, and ventral neck were shaved with electric clippers. The operative sites were scrubbed with povidone iodine soap, rinsed with alcohol, and painted with povidone iodine antiseptic. Each animal was placed on the surgical table with supplemental heat.

### Wound Procedure

A midline sternotomy was performed along the chest cavity. An incision (approximately 10 cm) was made through the skin along the anterior ventral midline. The sternum was exposed. Using an oscillating power saw and appropriate blade, the sternum was cut along the midline. Care was excised to avoid penetrating the thoracic cavity significantly beyond the sternum. Once the sternum had been incised, retractors were placed to expand the sternotomy opening. Gauze was immediately applied to the wound with light pressure to control immediate bleeding. The gauze was removed just prior to application of the treatment. The test material was prepared to a paste-like consistency and applied to the cut surface of the sternum and a stopwatch was started. The time of material application and the time to material hardening was recorded to the nearest minute. The bleeding was periodically evaluated until adequate hemostasis was reached. General observations were recorded approximately every minute. If no hemostasis was observed within a sufficient period, additional material may be applied. The amount of material used and the number of applications were recorded. Once hemostasis was completed in the sternum, the spleen was then wounded. Two separate incisions (2 to 3 mm deep by 4 to 6 cm long) were made in the parenchyma of the spleen over the anti-mesenteric surface using a BD Micro-Sharp 3.0 mm (15°). Gauze was applied immediately with light pressure to each incision in order to control immediate bleeding. The gauze was removed just prior to application of the treatment. The test material was applied to one wound and a stopwatch was started. The gauze was removed from the control incision and was left untreated for normal clotting. The time of hemostasis was recorded for the control incision. The time of material application and the time to material hardening were recorded to the Nearest minute. The bleeding was periodically evaluated until adequate hemostasis was reached for each incision. General observations were recorded approximately every minute. If no hemostasis was observed within a sufficient period, additional material may be applied. The amount of material used and the number of applications were recorded.

Once hemostasis was completed in the spleen, the liver was similarly wounded. Two separate incisions were made completely through the edge of a liver lobe, extending approximately 2-3 cm into the liver parenchyma. Gauze was applied immediately with light pressure to each incision to control immediate bleeding. The gauze was removed just prior to application of the treatment. The wound in the edge was opened, test material applied to one cut surface and the edges closed and held together with digital pressure for 30 seconds. After 30 seconds, pressure was released and a stopwatch was started. If the wound did not successfully adhere after application, an additional attempt to close (or approximate) the incision was made just prior to material hardening. The gauze was removed from the control incision and was left untreated for normal clotting. The time of hemostasis was recorded for the control incision. The time of material application, attempted wound adherences, and the time to material hardening were recorded to the nearest minute. The bleeding was periodically evaluated until adequate hemostasis was reached for each incision. General observations were recorded approximately every minute. If no hemostasis was observed within a sufficient period, additional material may be applied. The amount of material used and the number of applications were recorded.

Laboratory Observations (end-points)

I. Animals were monitored continuously throughout the procedure for temperature, heart rate, blood pressure, EKG, respiration rate, and PO2.
2. The animals were monitored for hemostasis time.

### Terminal Procedures

Following completion of hemostasis procedures, each animal was euthanized while under general anesthesia by an intravenous injection of a sodium pentobarbital based drug. No further evaluation of the wound was conducted. The carcass was discarded in accordance with standard operating procedures.

### Evaluations and Statistics

The end-points (hemostasis) were recorded and presented in tabular format for each treatment application and each organ. No statistical evaluation of the data was necessary for a procedure of this type

### 6. Results

### Surgical and Clinical Observations

The animals appeared clinically normal prior to surgery. For animal 5366, a small perforation to the right atrium occurred during the sternotomy procedure. The hole to the atrium was quickly repaired with sutures and additional fluids administered to maintain blood pressure. While this was an unanticipated event, it did not adversely affect the study objectives or outcome. Individual observations are presented in Appendix I.

### Body Weight Data

The body weights for these animals was acceptable for the study. Individual body weights are presented in Table 1.

### Hemostasis Data

All of the wounds resulted in a slow, oozing type bleeding wound, i.e.—no large, arterial bleeding. For the sternotomy wounds, the material was pressed into the cut surface of the wound. Hemostasis was achieved after an average of 12 minutes at the sternum defect site. Hemostasis was not achieved at the spleen and liver defect sites and the wounds continued to ooze throughout the observation period. With the continued oozing, the test material was floated out of or away from the cut surface of the wound. The material did not demonstrate any adherence to the liver or spleen. Additionally, hardening of the material was not obvious. This may have been due to dilution or mixing with blood at the wound site. Wound and material observations are presented in Tables 1 and 2.

### 7. Conclusion

Under the conditions of the study, the OsteoCrete would be an acceptable hemostatic agent in bone tissue. The test material did not show evidence of being an effective hemostatic agent in the organ parenchyma. Results and conclusions apply only to the test article tested. Any extrapolation of these data to other samples is the sponsor's responsibility. All procedures were conducted in conformance with good manufacturing practices and ISO 13485:2003.

### 8. Records

All raw data pertaining to this study and a copy of the final report are to be retained in designated NAMSA archive files.

### 9. References

Guide for the Care and Use of Laboratory Animals, Institute for Laboratory Animal Research, National Academy of Sciences (Washington: National Academy Press, 1996). ISO 10993: Biological Evaluation of Medical Devices, Part 4: Selection of tests for interactions with blood OLAW, Public Health Service Policy on Humane Care and Use of Laboratory Animals (NIH Publication). United States Code of Federal Regulation (CFR) 9: The Animal Welfare Act.

### 10. Protocol Changes

Any necessary changes to the protocol after sponsor approval or study initiation were documented and approved by the designated associate as protocol amendments. Copies were distributed to the sponsor and the raw data file.

TABLE 1
Surgical Observations

| | Body | |
|---|---|---|
| Animal- | Weight | |
| Number | (kg) | Surgical Observations |
| 5366 | 54 | The atrium was nicked during the sternotomy. The atrium was sutured |
| | | with 5-0 prolene prior to proceeding with the hemostasis procedure. |
| | | Mild to very slight oozing was noted at the sternotomy surgical defect |
| | | site for approximately 17 minutes after material application. At the |
| | | defect sites on the spleen, more oozing was noted from the test |
| | | incision than from the control incision. Very slight to slow oozing was |
| | | noted for approximately 16 minutes after material application. The |
| | | material did not harden at the defect site on the liver. Very slight to |
| | | brisk oozing was noted for approximately 30 minutes after material |
| | | application. Hemostasis was not achieved at the liver incision during |
| | | the 30 minute observation period. |
| 5365 | 52 | Slight to very slight oozing was noted along the sternotomy for |
| | | approximately 8 minutes following material application. At the defect |
| | | sites on the spleen, the material did not harden within 30 minutes of |
| | | application. Brisk oozing was noted for approximately 20 minutes after |
| | | material application, and hemostasis was not achieved during the 30 |
| | | minute observation period. The liver was not wounded. |

TABLE 2
Hemostasis Data

| | | Amount | | | | | |
|---|---|---|---|---|---|---|---|
| | | of | Time of | | | | |
| Animal | | Material | Material | Amount of Additional | | | Time of |
| Number | Location | Used | Hardening | Material Applied/Time | | | Hemostasis |
| 5366 | Sternum | 7 g | 10:58 AM | 10 g/ | 5 g | N/A | 11:06 AM |
| | | | | 10:55 AM | 11:00 AM | | |
| | Spleen | 2 g | 11:25 AM | 6 g | N/A | N/A | † |
| | | | | 11:23 AM | | | |
| | Liver | 5 g | N/A | 11:43 AM | N/A | N/A | ‡ |
| 5365 | Sternum | 15 g | 1:19 PM | 6 g | N/A | N/A | 1:21 PM |
| | | | | 1:15 PM | | | |
| | Spleen | 2 g | N/A | 3 g | 1:43 | N/A | ‡ |
| | | | | 1:30 PM | | | |
| | Liver | N/A | N/A | N/A | N/A | N/A | N/A |

NA Not Applicable
* The material did not initially harden due to bleeding. The material hardened in areas where not diluted by blood.
† Oozing was noted and hemostasis was achieved at II:46 AM.
‡ Hemostasis was not achieved within 30 minutes.

Having described the basic concept of the invention, it will be apparent to those skilled in the art that the foregoing detailed disclosure is intended to be presented by way of example only, and is not limiting. Various alterations, improvements, and modifications are intended to be suggested and are within the scope and spirit of the present invention. Additionally, the recited order of the elements or sequences, or the use of numbers, letters or other designations therefore, is not intended to limit the claimed processes to any order except as may be specified in the claims. Accordingly, the invention is limited only by the following claims and equivalents thereto.

All publications and patent documents cited in this application are incorporated by reference in their entirety for all purposes to the same extent as if each individual publication or patent document were so individually denoted.

The following is claimed as the present invention:

1. A method for providing hemostasis comprising:
supplying a dry potassium phosphate based hemostat mixture comprising:
monobasic potassium phosphate, a metal oxide, and a tertiary calcium phosphate, wherein the weight percent ratio of monobasic potassium phosphate to metal oxide is between about.3:1 and 1:1;
mixing the dry potassium phosphate based hemostat mixture with an aqueous solution forming an activated hemostat slurry;
applying a hemostasis-promoting amount of the activated based hemostat slurry to a site of bleeding, wherein the site of bleeding is a cut bone surface or exposed bone surface.

2. The method of claim 1, wherein the dry phase of the hemostat mixture further comprises: a sugar compound.

3. The method of claim 2, where the dry phase of the hemostat mixture further comprises: mono-sodium phosphate.

4. The method of claim 2, wherein the sugar compound is selected from the group consisting of: sugars, sugar derivatives, sugar replacements and combinations thereof.

5. The method of claim 2, wherein the sugar compound is selected from a group consisting of: sugars, sugar alcohols, sugar acids, amino sugars, sugar polymers glycosaminoglycans, glycolipids, sugar substitutes and combinations thereof.

6. The method of claim 2, wherein the sugar compound comprises sucrose.

7. The method of claim 1, wherein a sufficient amount of aqueous solution is mixed with the dry mixture to produce a paste-like slurry consistency.

8. The method of claim 1, wherein the amount of aqueous solution mixed with the dry mixture is between about 20 and 30 weight percent of the dry mixtures weight.

9. The method of claim 1, wherein the slurry is hand mixed.

10. The method of claim 1, wherein the activated further comprising at least one antibiotic.

11. The method of any of claims 1, wherein the activated composition further comprises one or more additional hemostatic agents selected from the group consisting of: collagen, collagen protein, thrombin, oxidized cellulose and combinations thereof

12. The method of claim 1, wherein the aqueous solution is a saline solution.

13. The method of claim 1, wherein the tertiary calcium phosphate is Ca₁₀(PO₄)₆(OH)₂.

14. The method of claim 13, wherein the metal oxide is MgO and wherein the dry hemostat mixture further comprises a sugar.

15. The method of claim 14, wherein the dry hemostat mixture further comprises mono-sodium phosphate.
